(19) United States
(12) Patent Application Publication
(10) Pub. No.: US 2007/0078436 A1
(43) Pub. Date: Apr. 5, 2007

(54) Balloon assisted apparatus and method for accessing an intervertebral disc

(76) Inventors: Andrea Y. Leung, Milpitas, CA (US); Hugues F. Malandain, Mountain View, CA (US); Erika I. Palmer, Menlo Park, CA (US); Paul M. Sand, San Carlos, CA (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/250,617

(22) Filed: Oct. 17, 2005

Related U.S. Application Data
(60) Provisional application No. 60/696,787, filed on Jul. 7, 2005.

(51) Int. Cl. A61M 31/00

(52) U.S. Cl. 604/509

(57) ABSTRACT

An apparatus includes a projection having a sharpened end portion configured to be inserted through an annulus of an intervertebral disc to define a distracted volume in the annulus. An expandable member is coupled to the projection and defines a longitudinal axis. The expandable member is configured to be expanded from a collapsed configuration to an expanded configuration in a direction substantially perpendicular to the longitudinal axis when at least a portion of the expandable member is disposed within the distracted volume of the annulus of the intervertebral disc. When the expandable member expands, it defines an expanded volume and defines a cross-sectional perimeter while in the expanded configuration. A cannula is configured to be at least partially inserted into the expanded volume. The cannula defines a cross-sectional outer perimeter having no portion extending outside of the cross-sectional perimeter of the expandable member while in its expanded configuration.

### CROSS-REFERENCE TO RELATED APPLICATION

This application claims priority to U.S. Provisional Application Ser. No. 60/696,787 entitled “Balloon Assisted Apparatus and Method for Accessing an Intervertebral Disc,” filed Jul. 7, 2005, the disclosure of which is incorporated herein by reference in its entirety.

### BACKGROUND

The invention relates generally to medical devices and procedures, and more particularly to a medical device for percutaneously accessing an intervertebral disc and creating a working channel for performing a medical procedure within an interior of the intervertebral disc.

There are a variety of medical devices configured to access bone or soft tissue within a body of a patient. For example, a scalpel can be used by a surgeon during invasive surgeries, while a bone drill can be used to percutaneously access the patient's body during a minimally invasive medical procedure. During an invasive surgery, a surgeon may make an excision with a scalpel, and then use another device to create a more visible working area within the patient's body. Such a device may be configured to spread apart bone and/or tissue to create visible access to an area within the patient's body.

In minimally invasive procedures, such as, for example a minimally invasive spinal procedure, a bone drill or other similar device may be used to create a path to a vertebra or disc within the patient's body. A device configured to further expand or spread bone or tissue may then be inserted through the path created with the drill. Other devices, such as a cannula, may also be necessary to provide a working channel for still other instruments. Thus, a variety of different medical devices may be required to initially access the patient's body and then create a working area to perform other medical procedures.

In both surgical and minimally invasive procedures, the process of gaining access to the interior of a patient's body can potentially result in damage to the bone or soft tissue being accessed. For example, the methods of cutting and/or drilling may remove portions of the bone or tissue, such that complete healing is not possible.

Thus, there is a need for a single apparatus and method that can be used to access a patient's body during a minimally invasive medical procedure, spread apart bone or tissue area as needed, and provide a working channel, with minimal damage to the surrounding bone or tissue.

### SUMMARY OF THE INVENTION

An apparatus includes a projection having a sharpened end portion configured to be inserted through an annulus of an intervertebral disc to define a distracted volume in the annulus. An expandable member is coupled to the projection and defines a longitudinal axis. The expandable member is configured to be expanded from a collapsed configuration to an expanded configuration in a direction substantially perpendicular to the longitudinal axis when at least a portion of the expandable member is disposed within the distracted volume of the annulus of the intervertebral disc. When the expandable member expands, it defines an expanded volume and defines a cross-sectional outer perimeter while in the expanded configuration. A cannula is configured to be at least partially inserted into the expanded volume. The cannula defines a cross-sectional outer perimeter having no portion extending outside of the cross-sectional outer perimeter of the expandable member while in its expanded configuration.

### BRIEF DESCRIPTION OF THE DRAWINGS

The present invention is described with reference to the accompanying drawings. In the drawings, like reference numbers indicate identical or functionally similar elements.

FIG. 1 is a side view of an apparatus according to an embodiment of the invention.

FIG. 2A is a side perspective view of a portion of the apparatus shown in FIG. 1.

FIG. 2B is a cross-sectional view taken along line 2B-2B in FIG. 2A.

FIG. 3 is side view of a portion of the apparatus shown in FIG. 1 in a collapsed configuration shown with a cross-sectional view of an intervertebral disc, according to an embodiment of the invention.

FIG. 4 is side of a portion of the apparatus shown in FIG. 1 in a collapsed configuration shown with a cross-sectional view of an intervertebral disc.

FIG. 5 is side view of a portion of the apparatus shown in FIG. 1 in an expanded configuration shown with a cross-sectional view of a portion of an intervertebral disc.

FIG. 6A is side view of a portion of the apparatus shown FIG. 1 in an expanded configuration shown partially in cross-section and with a cross-sectional view of a portion of an intervertebral disc.

FIG. 6B is a cross-sectional view taken along line 6B-6B in FIG. 6A.

FIG. 7 is side view partially in cross-section of a portion of the apparatus shown in FIG. 1 with a cross-sectional view of an intervertebral disc.

FIG. 8 is a side view of a portion of an apparatus in an expanded configuration shown partially in cross-section with a cross-sectional view of a portion of an intervertebral disc, according to another embodiment of the invention.

FIG. 9 is a side view of a portion of the apparatus of FIG. 8 shown partially in cross-section and with a cross-sectional view of a portion of an intervertebral disc.

FIG. 10 is a side view of a balloon constructed using known balloon construction technologies.

FIGS. 11A and 11B are side views of a portion of an apparatus including a balloon formed with known balloon construction technologies.

FIG. 12 is a partial cross-sectional view of a portion of the apparatus shown in FIG. 1.

FIG. 13 is a cross-sectional view of a portion of an apparatus according to an embodiment of the invention.

FIG. 14 is a cross-sectional view of a balloon during a heating process in a method of manufacturing according to an embodiment of the invention.

FIG. 15 is a cross-sectional view of the balloon of FIG. 14 during a stretching process in a method of manufacturing according to an embodiment of the invention.

FIG. 16 is a cross-sectional view of the balloon shown in FIGS. 14 and 15 after the stretching process.

FIG. 17 is a cross-sectional view of the balloon shown in FIGS. 14-16 during a molding process in a method of manufacturing according to an embodiment of the invention.

FIG. 18 is a cross-sectional view of the balloon shown in FIGS. 14-17 after the mold process.

FIG. 19 is a cross-sectional view of a balloon during a heating process in a method of manufacturing according to an embodiment of the invention.

FIG. 20 is a cross-sectional view of the balloon shown in FIG. 19 after a stretching process in a method of manufacturing according to an embodiment of the invention.

FIG. 21 is a cross-sectional view of the balloon shown in FIGS. 14-18 at the completion of the method of manufacturing in an expanded configuration according to an embodiment of the invention.

FIG. 22 is a cross-sectional view of the balloon shown in FIGS. 19-20 at the completion of the method of manufacture in an expanded configuration according to an embodiment of the invention.

### DETAILED DESCRIPTION

The apparatus and methods according to the invention provide for percutaneous access to the internal portion of an intervertebral disc. The apparatus is configured to penetrate the intervertebral disc with a speared stylet or projection making a small hole without cutting or shearing the fibre of the annulus. The projection is used in conjunction with an expandable member to create an access path or distracted volume within the intervertebral disc. A cannula can then be inserted into the distracted volume to provide a working channel to perform a variety of medical procedures within the interior of the intervertebral disc. The expandable member is constructed having a substantially uniform outer perimeter in a collapsed configuration, and sized such that it can follow the projection through the distracted volume created by the projection.

In one embodiment, an apparatus includes a projection having a sharpened end portion configured to be inserted through an annulus of an intervertebral disc to define a distracted volume in the annulus. An expandable member is coupled to the projection and defines a longitudinal axis. The expandable member is configured to be expanded from a collapsed configuration to an expanded configuration in a direction substantially perpendicular to the longitudinal axis when at least a portion of the expandable member is disposed within the distracted volume of the annulus of the intervertebral disc. When the expandable member expands, it defines an expanded volume and defines a cross-sectional outer perimeter while in the expanded configuration. A cannula is configured to be at least partially inserted into the expanded volume. The cannula defines a cross-sectional outer perimeter having no portion extending outside of the cross-sectional outer perimeter of the expandable member while in its expanded configuration.

In another embodiment, an apparatus includes a body having a first end and a second end. The second end of the body has a tapered point configured to penetrate an intervertebral disc and define a channel in the intervertebral disc. An expandable member is coupled to the body and spaced apart from the second end. The expandable member has an expanded configuration and a collapsed configuration. At least a portion of the expandable member is configured to be moved into a portion of the channel defined by the body. The expandable member is configured to be expanded in a substantially radial direction within an annulus of the intervertebral disc.

In another embodiment, a method includes selecting a width of a proximal end portion of a projection. The projection having a sharpened distal end portion and a length between the proximal end portion and the sharpened distal end portion. A taper angle of a wall between the proximal end portion and the sharpened distal end portion is selected such that the projection is configured to penetrate an annulus of an intervertebral disc. The projection is manufactured based on the selected width and the selected taper angle. The projection is coupled to an expandable member having a collapsed configuration and an expanded configuration associated with a width greater than the selected width.

In another embodiment, a method includes inserting a projection into at least a portion of an annulus of an intervertebral disc to define a distracted volume in the annulus. The projection has a first end and a second end and at least the second end of the projection tapers to a point. An expandable member is inserted into the distracted volume in the annulus, the expandable member is coupled to the projection and spaced apart from the second end. The expandable member is expanded in a direction substantially perpendicular to a longitudinal axis defined by the expandable member to define an expanded volume in the annulus.

‘The term “expandable member is used here to mean a component of the apparatus being configured to move from a collapsed configuration to an expanded configuration. The expandable member can be, for example, a balloon configured to expand in a direction substantially perpendicular to an axis defined by the expandable member.

The term “cannula” is used here to mean a component of the apparatus having one or more passageways configured to receive a medical device therethrough and provide access to an interior portion of an interveterbral disc. For example, the cannula can be substantially tubular. The cannula can be a variety of different shapes and size, such as having a round or octagonal outer perimeter.

The term “projection” is used here it mean a component of the apparatus that is configured to penetrate an intervertebral disc and create an opening within the intervertebral disc. The projection can include, for example, a sharpened tip portion or a wall having a tapered or angled portion.

The term “distracted volume” is used here to mean that portion of an annulus of an intervertebral disc that is penetrated by the projection. For example, the distracted volume is an opening created within the intervertebral disc that can be expanded and then will contract to a substantially closed condition with minimal permanent defects to the intervertebral disc after the projection is removed.

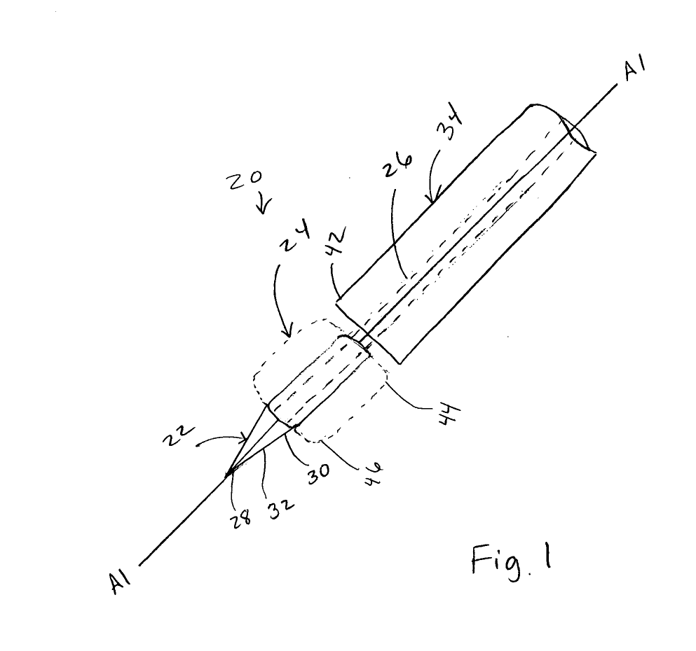

FIG. 1 illustrates an apparatus 20 according to an embodiment of the invention. Apparatus 20 includes a projection 22, an elongate portion 26, an expandable member 24 and a cannula 34. The projection 22 includes a proximal end portion 30 and a sharpened distal end portion 28. A wall 32 extends between the proximal end portion 30 and the distal end portion 28. The wall 32 defines a taper angle sufficient such that projection 22 can penetrate an annulus of an intervertebral disc. The projection 22 can be coupled to either the expandable member 24 or the elongate portion 26. Alternatively, the projection 22 and elongate portion 26 can be unitarily formed as a single component.

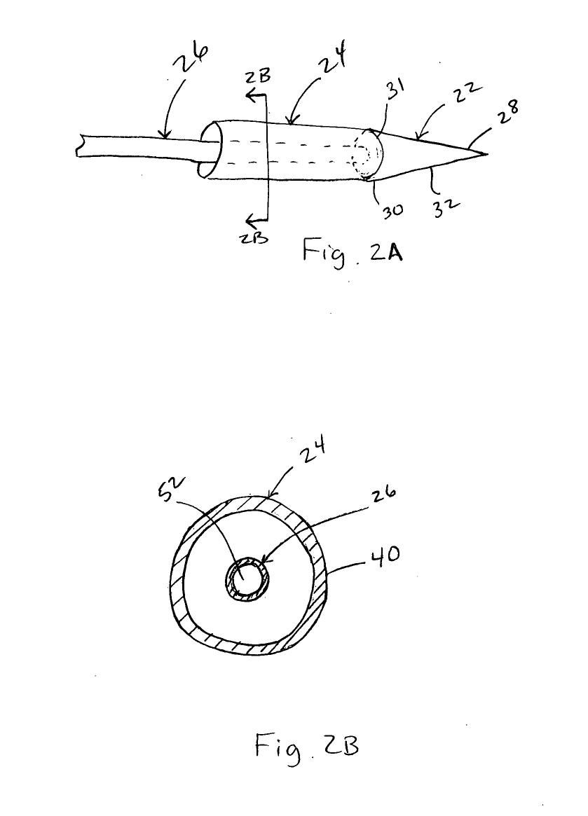

The expandable member 24 can be coupled to the projection 22 and/or the elongate portion 26. The expandable member 24 includes a proximal end portion 44 and a distal end portion 46 and is configured to move from a collapsed configuration to an expanded configuration. For example, the expandable member 24 can be a balloon configured to be inflated with pressurized fluid or gas (e.g., air, water) to expand a cross-sectional outer perimeter 40 (shown in FIG. 2B) of expandable member 24. The elongate portion 26 can include a channel 52 (see FIGS. 2B, 6B and 12) and one or more apertures 48 (see FIG. 12) disposed on elongate portion 26 that communicate with the channel 52. The pressurized fluid or gas used to expand the expandable member 24 can be communicated to an inner cavity 50 in the expandable member 24 through the one or more apertures 48. The pressurized fluid or gas can be controlled with a pressure relief valve (not shown) to ensure the proper level of pressure is provided. The expandable member 24 is constructed such that it will expand substantially radially and uniformly along a portion of a longitudinal length of the expandable member 24. In other words, at least a portion of the expandable member 24 can expand substantially perpendicularly to an axis A1 (see FIG. 1) defined by the expandable member 24. A method of manufacturing an embodiment of an expandable member will be described in more detail below. The configuration of projection 22 will depend on a variety of factors, including the desired size of an opening or distracted volume within the annulus of the intervertebral disc. The manufacture of a projection 22 will include choosing the desired size of an outer perimeter 31 (or width) of the proximal end portion 30 (see FIG. 2A), and a taper angle of the wall 32, both of which will affect the size of the distracted volume to be created within the annulus of the intervertebral disc. The projection 22 can be, for example, substantially cone shaped as shown in FIG. 2A. The selected size of the outer perimeter 31 of the proximal end portion 30 of the projection 22 will also depend on the size of an outer perimeter 40 (shown in FIG. 2B) of the expandable member 24 in its collapsed configuration. For example, in some embodiments, the outer perimeter 31 of the proximal end portion 30 of the projection 22 should be no greater than the outer perimeter 40 of the expandable member 24 in its collapsed configuration. In some embodiments, the outer perimeter 31 of the proximal end portion 30 of the projection 22 can be between 2.0 mm and 3.0 mm (0.08″-0.12″). In some embodiments, the taper angle of the wall 32 of the projection 30 will be between 11° and 12°.

The expandable member 24 in its collapsed configuration can include an outer perimeter 40 that is substantially the same size as the outer perimeter 31 of the proximal end portion 30 of the projection 22. This allows the expandable member 24 to follow the projection 22 through the annulus of the intervertebral disc as the projection penetrates the intervertebral disc. If the outer perimeter 40 of the expandable member 24 is too large, the path created by the projection 22 within the annulus may be too small to permit the expandable member 24 to pass through it. If the outer perimeter 40 of the expandable member 24 in its collapsed configuration is too small or narrow, and a cannula is not used, the proximal end portion 30 of the projection 22 may drag along the fibre of the annulus when being removed from the intervertebral disc.

In its expanded configuration, the outer perimeter 40 of the expandable member 24 should be larger in size than the outer perimeter 31 of the proximal end portion 30 of the projection 22, such that when the expandable member 24 is expanded it will expand the distracted volume created by the projection 22. Thus, the relationship between the outer perimeter 31 of the proximal end portion 30 of the projection 22 and the outer perimeter 40 of the expandable member 24 is an important factor in determining the size and shape of those components.

In some embodiments, the expandable member 24 includes a substantially uniform outer perimeter 40 and a cavity 50 with a non-uniform diameter in its collapsed configuration, as shown in FIG. 12. In such an embodiment, the expandable member 24 in its collapsed configuration includes a first portion 54, a second portion 56 and a third portion 58. The second portion 56 has a wall thickness that is thinner than a wall thickness of the first portion 54 and a wall thickness of the third portion 58. In addition, the cavity 50 associated with the second portion 56 includes a portion having a larger inner diameter than an inner diameter of the cavity 50 associated with the first portion 54 and an inner diameter of the cavity 50 associated with the third portion 58.

In an expanded configuration, the outer perimeter 40 of the expandable member 24 varies along the longitudinal length (see FIG. 6A). For example, the second portion 56 of the expandable member 24 has a larger outer perimeter (or diameter) than the first portion 54 and the third portion 58.

The cannula 34 includes a distal end portion 42, at least one channel 36 (shown in FIGS. 6A and 6B), and defines a cross-sectional outer perimeter 38 (shown in FIG. 6B). Cannula 34 can be any known cannula having a suitable cross-sectional outer perimeter to use in conjunction with the expandable member 24 to be discussed in more detail below. Thus, cannula 34 can be a variety of different shapes and sizes.

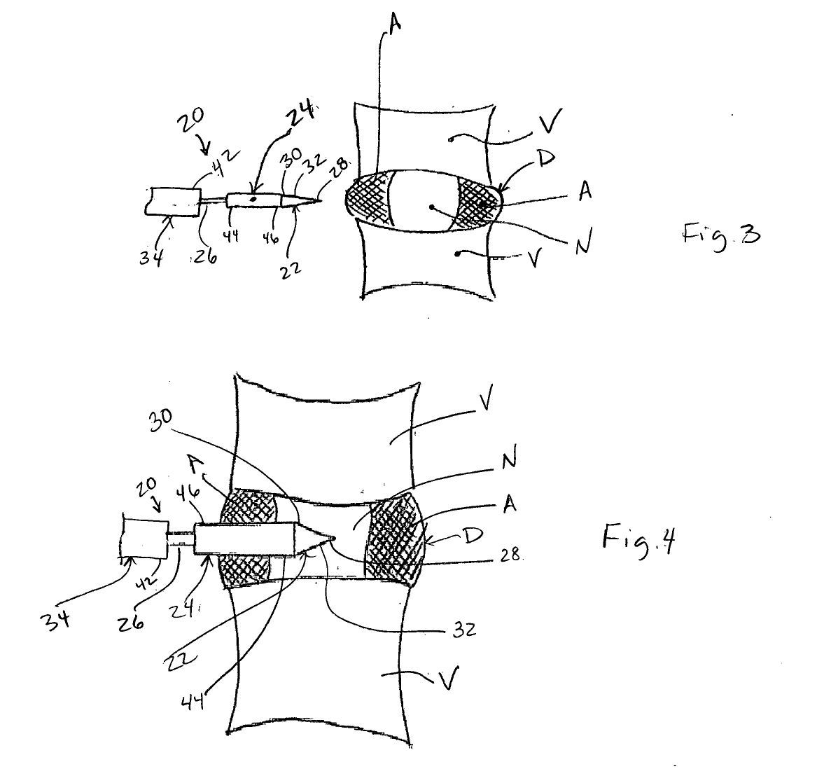

Referring now to FIGS. 3 through 7, apparatus 20 is configured to percutaneously access an annulus A of an intervertebral disc D and create an access path to the interior portion or nucleus N of the intervertebral disc. In use, the expandable member 24 is placed in a collapsed configuration, as shown in FIG. 3. The projection 22 can be used to penetrate the annulus A of the intervertebral disc D positioned between vertebras V. The projection 22 can be pushed or stabbed into the annulus A (see FIG. 4) with a motion similar to how a dagger or pin would be used, rather than with a cutting motion (e.g., a back and forth motion). The projection 22 creates an opening or a distracted volume within the annulus A of the intervertebral disc D. Because the projection 22 is configured such that it penetrates the annulus A, rather than cuts it, potential shear forces exerted on the fibre of the annulus A are substantially reduced, if not eliminated.

When penetrating the annulus A, the cannula 34 can be coupled to the elongate portion 26 or expandable member 24, and positioned distally from the expandable member 24. Alternatively, the cannula 34 may not be coupled to the elongate portion 26 or the expandable member 24 when the projection 22 penetrates the annulus A. In such an embodiment, the cannula 34 can be positioned over the elongate portion 26 after the annulus A has been penetrated.

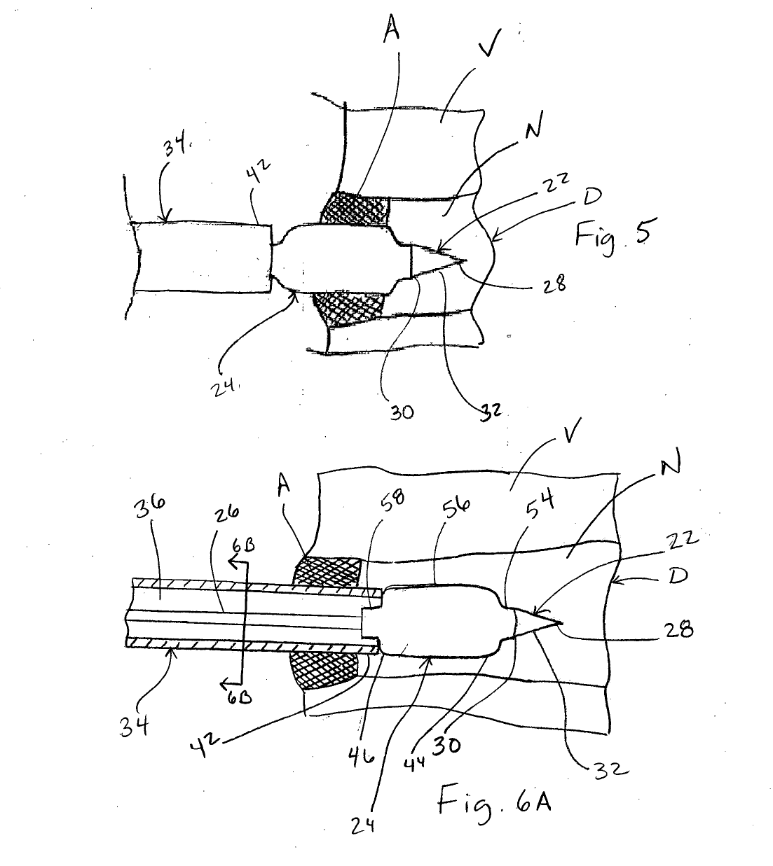

After the projection 22 has penetrated the annulus A, and when the expandable member 24 is positioned within the annulus A, the expandable member 24 can be moved to its expanded configuration, as shown in FIG. 5. The expansion of expandable member 24 expands or dilates the distracted volume to create a larger opening in the annulus A, such that the cannula 34 can be passed through the expanded distracted volume. The cannula 34 can then be positioned such that the distal end portion 42 of the cannula 34 is positioned proximally from the proximal end portion 44 of the expandable member 24 or contacting the proximal end portion 44 of the expandable member 24, as shown in FIGS. 5 and 6A. As stated above, the expandable member 24 is configured such that at least a portion of the expandable member 24 expands substantially radially and uniformly along the longitudinal length of the expandable member 24. For example, as shown in FIGS. 5 and 6A, the second portion 56 expands substantially radially and uniformly. This uniform expansion exerts force substantially uniformly within the distracted volume of the annulus A, which further reduces any shearing effects to the surrounding fibre of the annulus A.

While the expandable member 24 is in its expanded configuration, the apparatus 20 can be pushed further through the annulus A, such that the expandable member 24 is positioned substantially within the nucleus N of the intervertebral disc D, as shown in FIG. 6A. The path created by the projection 22 and the expandable member 24 allows the cannula 34 to be inserted through the annulus A, such that the distal end portion 42 of the cannula 34 is positioned at least partially within the nucleus N of the intervertebral disc, as shown in FIG. 6A. As stated above, the cannula 34 can follow closely behind the expandable member 24 or contacting the proximal end portion 44 of the expandable member 24, as the expandable member 24 is being pushed through the distracted volume. The cross-sectional outer perimeter 38 of the cannula 34 is sized such that no portion of the cannula 34 extends outside of the cross-sectional outer perimeter 40 of the expandable member 24 when the expandable member 24 is in its expanded configuration, as shown in FIG. 6A. This dimensioning ensures that the cannula 34 can fit within the path or expanded volume created by the expandable member 24 in the annulus A. This also reduces shear forces being exerted on the annulus A as the cannula 34 is being moved through the annulus A of the intervertebral disc D.

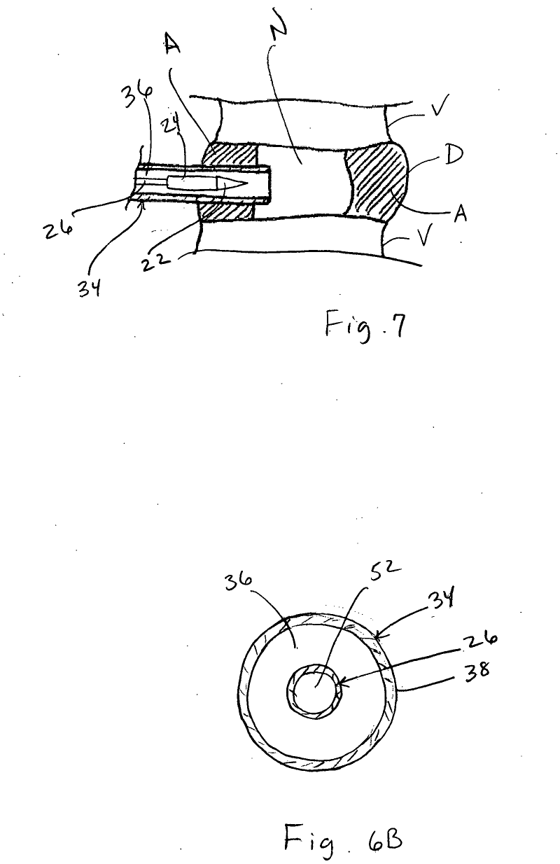

After the cannula 34 is positioned at a desired location within the intervertebral disc D with a portion of the cannula 34 protruding into the nucleus N as shown in FIG. 6A, the expandable member 24 can be moved to its collapsed configuration. In the collapsed configuration, the perimeter 40 of the expandable member 24 and the width of the projection 22 are smaller than the passageway 36 of cannula 34. This allows the projection 22 and the expandable member 24 to be withdrawn or removed from the intervertebral disc D through the passageway 36, as shown in FIG. 7. After the projection 22 and the expandable member 24 have been removed from the intervertebral disc, the nucleus N of the intervertebral disc D can be accessed via the channel 36 of the cannula 34 to perform a variety of different medical procedures. After a medical procedure has been performed on the intervertebral disc D, the cannula 34 can then be removed from the patient's body. Because the annulus A was penetrated with a stabbing force, rather than a cutting procedure, minimal tissue or fibre of the annulus A will have been removed and/or damaged. Therefore, the distracted volume created in the annulus A will substantially completely close, minimizing defects to the surrounding tissue or fibre of the annulus A.

FIGS. 8 and 9 illustrate an embodiment of an apparatus 120 according to another embodiment of the invention. The apparatus 120 is substantially similar in function and structure as apparatus 20. Apparatus 120 includes a projection 122, an elongate portion 126, an expandable member 124 and a cannula 134. The apparatus 120 penetrates the annulus A of an intervertebral disc D as previously described, but in this embodiment, the expandable member 124 is shorter in length. The shorter length of the expandable member 124 may require incremental expansion of a distracted volume along a width of the annulus A.

In use, the apparatus 120 is at least partially inserted into the annulus A of an intervertebral disc D, with the projection 122 penetrating the annulus A and creating a distracted volume as described previously. The expandable member 124 follows behind the projection 122 in a collapsed configuration. The expandable member 124 is then expanded, thereby expanding the distracted volume of a portion of the annulus A, as shown in FIG. 8. The expandable member 124 is then moved back to a collapsed configuration so that the projection 122 can be pushed further into the annulus A of the intervertebral disc D. The expandable member 124 is again expanded, thereby expanding the distracted volume created by the projection 122, as shown in FIG. 9. This process of pushing the apparatus 120 through the width of annulus A of the intervertebral disc D, expanding the expandable member 124, and then collapsing the expandable member 124, can be repeated until at least a portion of the apparatus 120 is positioned within the nucleus N of the intervertebral disc D.

As with the previous embodiment, the cannula 134 can be disposed over the elongate portion 126 and inserted into the expanded distracted volume created by expanding the expandable member 124, as shown in FIGS. 8 and 9. Alternatively, the cannula 134 can be placed over the elongate portion 126 after the distracted volume is expanded. Thus, the cannula 134 can provide a working channel to the nucleus N of the intervertebral disc D.

The apparatus for any of the embodiments may be constructed with any suitable material used for such a medical device. For example, the projection can be constructed with a biocompatible material, such as stainless steel. The elongate portion and the cannula can be constructed with a biocompatible metal, such as stainless steel, or suitable plastic materials, such as various polymers.

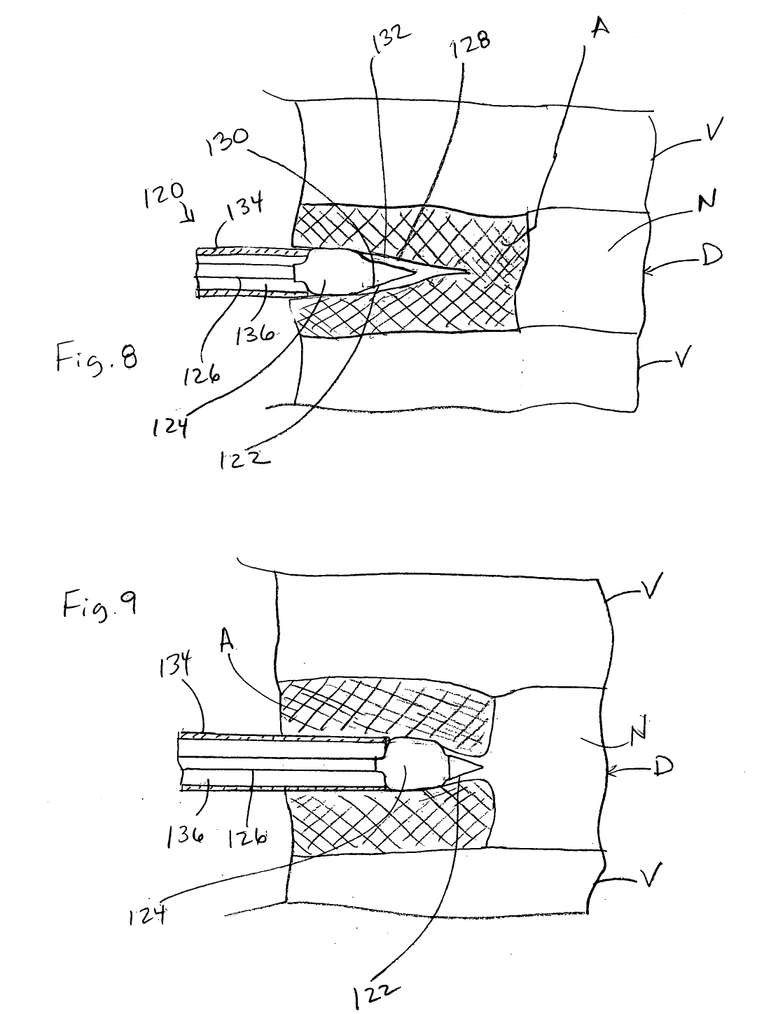

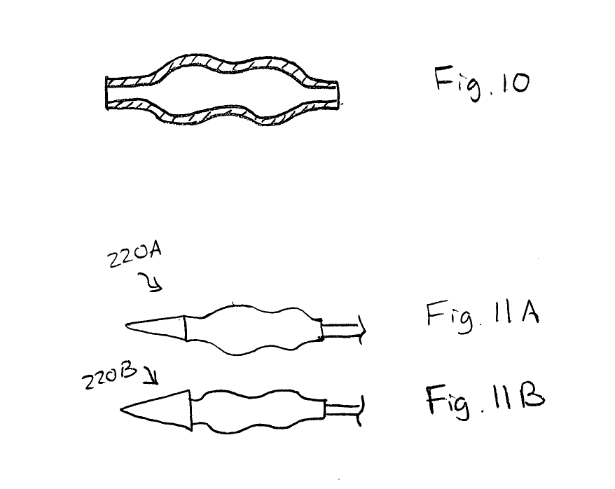

The expandable member can be constructed of suitable plastic or rubber materials, such as various polymers. To obtain the desired uniform radial expansion of at least a portion of the expandable member, the expandable member can be formed with a substantially uniform profile in a collapsed configuration. A process or method of constructing such an expandable member having the characteristics as described above will now be described. Current balloon technology includes blowing and forming tubing inside a mold with a specific shape, resulting in a non-cylindrical outer diameter of the balloon in a collapsed configuration. FIG. 10 illustrates an example of a balloon constructed with current balloon technology having a non-uniform outer diameter. If this current technology were used to construct the expandable member included in the medical device 20 (120), the result would be an apparatus 220A or 220B, for example, as shown in FIGS. 11A and 11B. The apparatus 220A (and 220B) would not be ideal for inserting and/or removing a projection (or other sharp instrument) from the body of the patient because of its non-uniform profile. As described previously, the relationship between the size and shape of the outer perimeter of the expandable member proximate the projection, and the size and shape of the outer perimeter of the projection allows for a smooth transition between the projection and the expandable member.

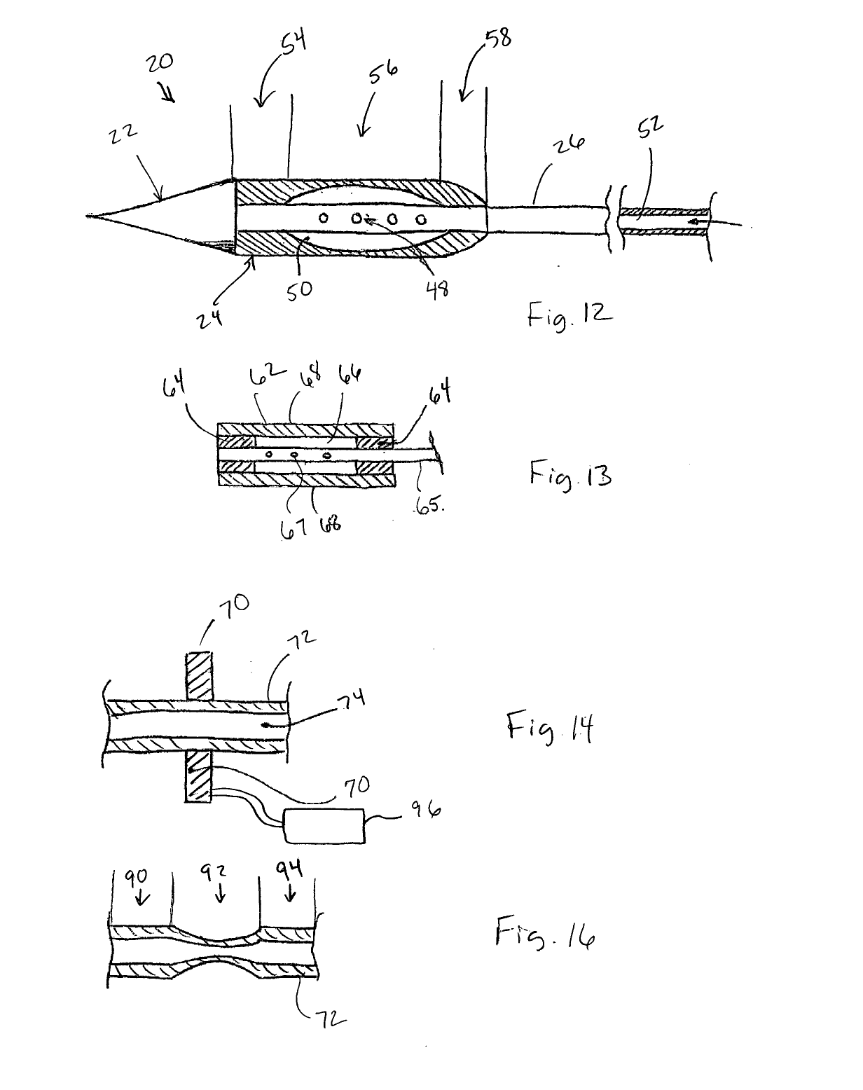

To solve these problems, an expandable member (hereinafter referred to as a balloon) can be formed having a substantially constant outer diameter in its collapsed configuration and used as the expandable member on the apparatus 20 (120). One method of forming a balloon having a constant outer diameter includes creating an internal cavity on the balloon. The method includes placing a thin sheath 62 of suitable material over sleeves 64, as shown in the cross-sectional view of FIG. 13. The sleeves 64 are bonded to the sheath 62 using known bonding methods. This allows the resulting balloon to have weaker and/or relatively more compliant walls at predetermined locations 68 where the balloon material is not supported by the sleeves 64. An internal cavity 66 is created, as shown in FIG. 13, that can be subsequently inflated in use to move the balloon from a collapsed configuration to an expanded configuration as previously described.

An alternative method of forming a balloon having a constant outer diameter includes producing weakened areas along the length of the balloon, without physically attaching a component such as the sleeves 64 to the balloon material. To achieve this, a heating process is performed. As shown in FIG. 14, a heating device 96 can be used to heat a selected portion of a tubular member to a temperature greater than another portion of the tubular member. For example, the heating device 96 can include one or more heat blocks 70 that are placed radially at selected locations along a tubular member 72 defining a lumen 74, as shown in FIG. 14. The heating process will weaken the selected portion of the tubular body 72 where the heat block(s) 70 are disposed.

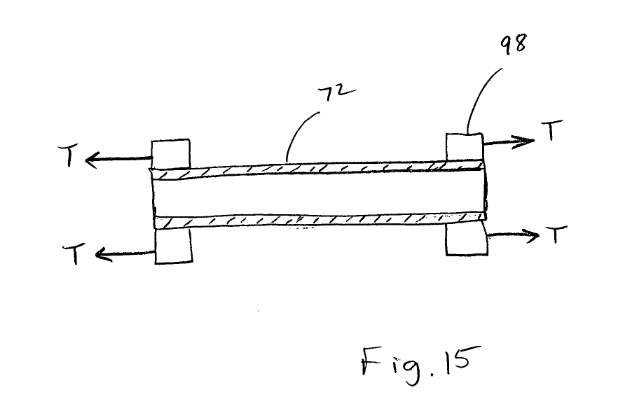

The next step is to stretch the tubular body 72 that was previously heated, as shown in FIG. 15. The tubular body 72 is placed in a stretching station 98 where opposite ends of the tubular body 72 are held. The stretching station 98 can have, for example, a pair of clamps that are moveable on a linear track. The stretching process exerts a tension force T on the opposite ends of the tubular body 72. During the stretching process, the weakened areas will concave inwardly towards the center of the tubular body 72, as shown in FIG. 16. As shown in FIG. 16, the tubular body 72 will include a first portion 90, a second portion 92 and a third portion 94. After the stretching process, the second portion 92 includes the pre-selected location on the outer perimeter of the tubular body (i.e., where the heat blocks were placed) and has a smaller inner diameter than an inner diameter of the first portion 90 and an inner diameter of the third portion 94. In addition, at least a portion of the outer diameter of the second portion 92 will be smaller than an outer diameter of the first portion 90 and an outer diameter of the third portion 94 after the stretching process.

Next, a mold process can be performed. The tubular body 72 is held within a mold 76, as shown in FIG. 17. The mold 76 can be configured, for example, to hold the tubular body 72 within a cylindrical shaped mold cavity. Pressurized air P (or other suitable gas) can then be blown into the cavity 74, as shown in FIG. 17. A plug 78 can be used to trap the pressurized air within the cavity 74. The mold process cools the tubular body 72, such that the tubular body 72 conforms to the mold cavity. The resulting form of the balloon is illustrated in FIG. 18. As shown, the inner diameter of the tubular body 72 is non-constant along the longitudinal length of the tubular body 72. Specifically, after the molding process, the second portion 92 includes a larger inner diameter than an inner diameter of the first portion 90 and an inner diameter of the third portion 94. The outer diameter of the tubular body 72 along the longitudinal length of tubular body 72, however, is substantially constant after the mold process. The second portion 92 includes the weakened areas of the wall of the tubular body 72, which are less thick than the wall of the first portion 90 and the wall of the third portion 94. The weakened areas of the wall of the second portion 92 are more flexible than the wall included in the first portion 90 and the third portion 94. When the tubular body 72 is later inflated, the weakened areas of the second portion 92 will expand further from a centerline C of the tubular body 72 than the walls of the first portion 90 and the third portion 94, as shown in FIG. 21. Thus, a substantially uniform and cylindrical outer diameter associated with the second portion 92 can be produced in an inflated or expanded configuration, as well as in a collapsed configuration (see FIG. 18).

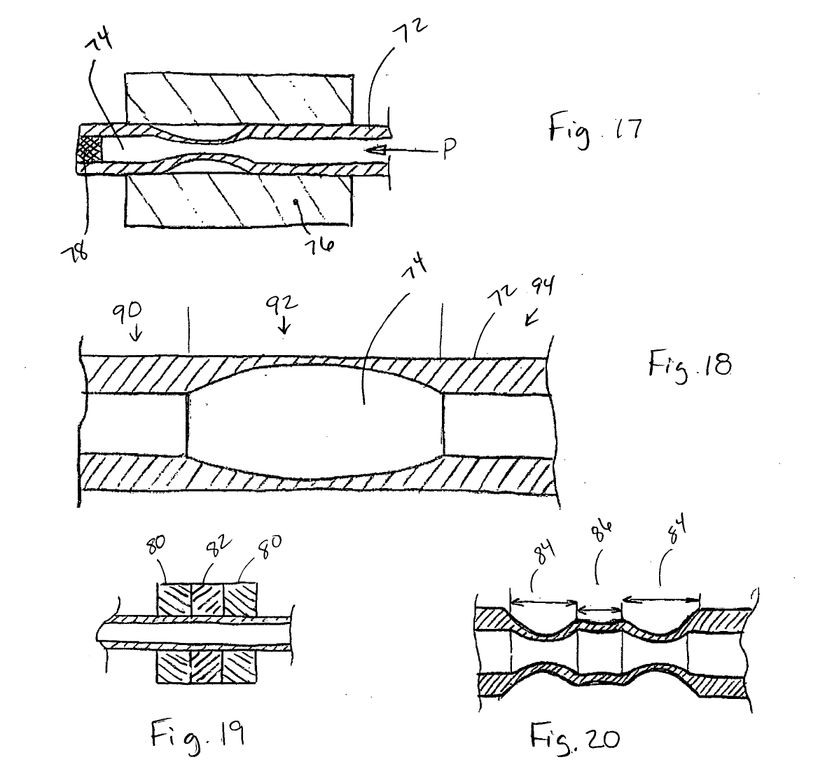

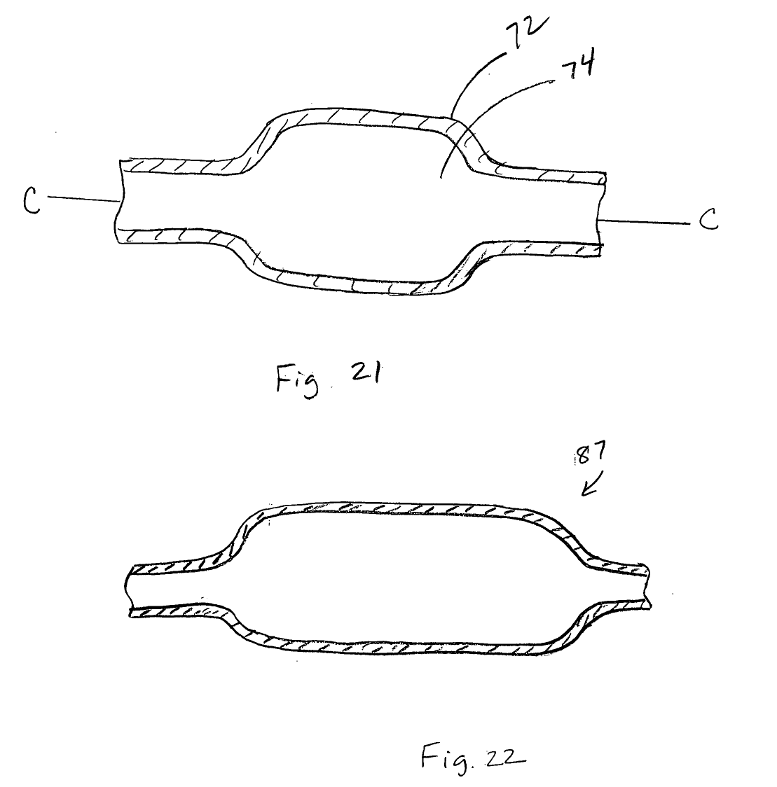

The method described above can be performed using a variety of different heat blocks of various shapes and sizes. The heat blocks can also be configured with various temperatures, and positioned at various locations along the balloon surface, depending on the desired resulting balloon shape and size. Thus, a variety of different outer perimeters or diameters of the balloon can be achieved based on the different weakened areas created. For example, FIG. 19 illustrates the use of multiple heat blocks having varied temperatures. In this example, the heat blocks 80 are at a higher temperature than the heat block 82. Therefore, after the stretching process, the balloon will have varied weakened areas along its length, as shown in FIG. 20. The weakened areas 84 produced by heat blocks 80 were heated to a higher selected temperature, and the weakened area 86 produced by heat block 82 was heated to a lower selected temperature. After the molding process, and upon subsequent inflation of the balloon, the weakened areas 84 will stretch more than the weakened area 86 resulting in a balloon 87 (see FIG. 22) that will be similar to the balloon (tubular body 72) illustrated in FIG. 21, except that the length of the cylindrical portion in the inflated or expanded configuration will be longer. This configuration may be desired, for example, to provide more strength to the balloon 87 in area 86.

Although the above description of manufacturing a balloon focused on producing a balloon with a constant outer diameter, the same methods can be used to produce a balloon having a constant outer perimeter. For example, a non-circular balloon may be desired for a particular application. In such an embodiment, the shape and size of the perimeter of the balloon can be constructed to match the shape and size of the projection as described above. In addition, it should be understood that the methods of manufacturing a balloon described above can be used to manufacture the expandable member 24 (124) included on the medical devices 20 (120) described herein. As described previously, a constant outer diameter or outer perimeter of the expandable member 24 (124) is desired to provide a smooth entrance through an intervertebral disc following the projection 22 (122).

### CONCLUSION

While various embodiments of the invention have been described above, it should be understood that they have been presented by way of example only, and not limitation. The invention has been particularly shown and described with reference to specific embodiments thereof, but it will be understood that various changes in form and details may be made.

For example, the apparatus can be used with or without the cannula described herein. The various components of the apparatus, including the cannula, the elongate portion, the expandable member and the projection can each be constructed having various sizes and shapes. In addition, although the apparatuses and methods described herein focused on the use of the apparatus on an intervertebral disc, it should be understood that the apparatus and methods described can be used to provide percutaneous access to other areas of a patient's body.

What is claimed is:

1. An apparatus, comprising:
a projection having a sharpened end portion configured to be inserted through an annulus of an intervertebral disc to define a distracted volume in the annulus;
an expandable member coupled to the projection and defining a longitudinal axis, the expandable member configured to be expanded from a collapsed configuration to an expanded configuration in a direction substantially perpendicular to the longitudinal axis when at least a portion of the expandable member is disposed within the distracted volume of the annulus of the intervertebral disc to define an expanded volume, the expandable member defining a cross-sectional perimeter while in the expanded configuration; and
a cannula configured to be at least partially inserted into the expanded volume, the cannula defining a cross-sectional outer perimeter having no portion extending outside of the cross-sectional perimeter of the expandable member while in its expanded configuration.

2. The apparatus of claim 1, wherein the sharpened end portion of the projection is a pointed tip.

3. The apparatus of claim 1, wherein the expandable member is a balloon, the balloon configured to expand uniformly in at least the direction substantially perpendicular to the longitudinal axis.

4. The apparatus of claim 1, wherein the cannula defines a passageway configured to provide access to an interior portion of the intervertebral disc.

5. A method, comprising:
selecting a width of a proximal end portion of a projection, the projection having a sharpened distal end portion, the projection having a length between the proximal end portion and the sharpened distal end portion;
selecting a taper angle of a wall between the proximal end portion and the sharpened distal end portion such that the projection is configured to penetrate an annulus of an intervertebral disc;
manufacturing the projection based on the selected width and the selected taper angle; and
coupling the projection to an expandable member having a collapsed configuration and an expanded configuration associated with a width greater than the selected width.

6. The method of claim 5, wherein the selecting the width of the proximal end portion includes selecting the width from the range 2.0 mm to 3.0 mm.

7. The method of claim 5, wherein the selecting the taper angle of the sharpened distal end portion includes selecting the taper angle from the range 1° to 12°.

8. A method, comprising:
inserting a projection into at least a portion of an annulus of an intervertebral disc to define a distracted volume in the annulus, the projection having a first end and a second end, at least the second end of the projection tapering to a point;
inserting an expandable member into the distracted volume in the annulus, the expandable member being coupled to the projection and spaced apart from the second end; and
expanding the expandable member in a direction substantially perpendicular to a longitudinal axis defined by the expandable member to define an expanded volume in the annulus.

9. The method of claim 8, further comprising:
inserting a cannula into the expanded volume of the annulus of the intervertebral disc, the cannula having an outer diameter no greater than a diameter of the expandable member in an expanded configuration;
collapsing the expandable member to a collapsed configuration, the diameter of the expandable member in the collapsed configuration being less than a diameter of an internal passageway defined by the cannula; and
removing the expandable member and the projection from the intervertebral disc through the internal passageway.

10. The method of claim 8, wherein the expanding the expandable member includes inflating the expandable member.

11. The method of claim 8, further comprising:
inserting a cannula at least partially into the expanded volume of the annulus of the intervertebral disc, the cannula having an outer diameter no greater than a diameter of the expandable member in an expanded configuration;
collapsing the expandable member to a collapsed configuration;
inserting the projection into a second portion of the annulus to define a second distracted volume in the annulus after the collapsing the expandable member;
inserting the expandable member into the second distracted volume in the annulus; and
expanding the expandable member in a direction substantially perpendicular to the longitudinal axis to define a second expanded volume in the annulus.

12. An apparatus, comprising:
a body having a first end and a second end, the second end of the body having a tapered point configured to penetrate an intervertebral disc and define a channel in the intervertebral disc; and
an expandable member coupled to the body and spaced apart from the second end, the expandable member having an expanded configuration and a collapsed configuration, at least a portion of the expandable member configured to be moved into a portion of the channel defined by the body, the expandable member configured to be expanded in a substantially radial direction within an annulus of the intervertebral disc.

13. The apparatus of claim 12, further comprising: a cannula, the cannula defining a passageway having a diameter, the diameter of the passageway being no greater than a diameter of the channel in the intervertebral disc when the expandable member is in its expanded configuration, the cannula configured to be moved into the intervertebral disc without increasing the diameter of the channel in the intervertebral disc.

14. The apparatus of claim 12, wherein the second end of the body is a substantially conical configuration, the slope of the cone being from 1° to 12°.

15. The apparatus of claim 12, wherein the body and the expandable member are configured to be withdrawn from the intervertebral disc through the passageway of the cannula.
